(19) United States
(12) Patent Application Publication
(10) Pub. No.: US 2023/0355199 A1
(43) Pub. Date: Nov. 9, 2023

(54) SYSTEMS AND METHODS FOR EVALUATION AND PREDICTION OF RISK OF MALIGNANT EDEMA AFTER STROKE

(71) Applicant: Washington University, St. Louis, MO (US)

(72) Inventors: Rajat Dhar, St. Louis, MO (US); Jin-Moo Lee, St. Louis, MO (US); Yasheng Chen, St. Louis, MO (US); Daniel Marcus, St. Louis, MO (US); Hossein Mohammadian Foroushani, St. Louis, MO (US)

(21) Appl. No.: 18/311,338

(22) Filed: May 3, 2023

Related U.S. Application Data
(60) Provisional application No. 63/364,124, filed on May 4, 2022.

(51) Int. Cl. A61B 6/00; A61B 6/03; G16H 50/20

(52) U.S. Cl. CPC A61B 6/5217; A61B 6/032; A61B 6/501; G16H 50/20

(57) ABSTRACT

A system for determining a likelihood of future malignant edema occurring in a patient includes an input, a processor coupled to the input, and a memory coupled to the processor. The memory includes instructions that program the processor to receive, through the input, computed tomography (CT) scans of the patient after occurrence of a stroke in the patient, determine at least one cerebrospinal fluid (CSF) metric from the CT scans, and determine a likelihood of a future malignant edema occurring in the patient based at least in part on the at least one CSF metric.

### CROSS REFERENCE TO RELATED APPLICATIONS

This application claims priority to U.S. Provisional Patent Application Ser. No. 63/364,124, filed May 4, 2022, which is hereby incorporated herein by reference in its entirety.

### STATEMENT REGARDING FEDERALLY SPONSORED RESEARCH & DEVELOPMENT

This invention was made with government support under NS099440, NS085419 and NS098577 awarded by the National Institutes of Health. The government has certain rights in the invention.

### BACKGROUND

This disclosure relates generally to evaluation of patients after a stroke. More specifically, this disclosure relates to evaluating patients after a stroke and evaluating the risk of malignant edema.

Cerebral edema develops in the hours to days after acute ischemic stroke and may result in midline shift and cerebral herniation. Even though a leading cause of death and deterioration, especially for strokes due to large vessel occlusion (LVO), only a small proportion of all stroke patients will develop this life-threatening complication. As deterioration is usually delayed by a few days after stroke, an important opportunity for early detection and intervention exists. Decompressive hemicraniectomy (DHC), if performed prior to deterioration and within 48 hours, dramatically reduces mortality and improves chances of functional recovery. Accurately predicting which hemispheric stroke patients will go on to develop malignant edema is therefore of vital importance in acute stroke care.

As key mediators remain incompletely understood, few interventions currently exist to mitigate cerebral edema. One of the major limitations in studying edema is the need for an accurate means of quantifying its formation in the early stages after stroke. Midline shift is a crude measure that does not adequately capture edema as it develops over the first 24-48 hours after stroke, but only captures its delayed and decompensated phenotype. Furthermore, labeling edema only when it leads to deterioration (i.e., malignant edema) obscures a continuum of injury severity that is seen across almost all LVO stroke patients.

One of the hallmarks of evolving brain edema is tissue hypoattenuation. This can be captured by the progressively decreasing density (measured in Hounsfield Units, HU) of infarcted tissue on non-contrast computed tomography (NCCT) imaging. NCCT is readily available and routinely performed in almost all stroke patients, both acutely on presentation and frequently at follow-up. It affords an accessible means of serially assessing edema as it develops in the days after stroke. However, measurement of the total lesional hypodensity volume encompasses both infarcted tissue and associated edema, with relative proportions varying across patients. One known imaging method disentangles the contribution of edema to subacute lesion volume and quantify the progression edema after stroke. Net water uptake (NWU) evaluates the relative density of the ischemic tissue compared to a contralateral homologous region; increasing NWU on admission NCCT has been associated with longer time from stroke onset to imaging and poor collateral status. NWU has also exhibited promise in quantifying edema progression, rising more in those with malignant outcomes and in those without successful recanalization. Therefore, it has emerged as one of the most promising biomarkers of edema after stroke, with a wide array of potential applications across LVO cohorts.

However, implementation of NWU measurement from serial CTs in large stroke cohorts faces several challenges. For example, its assessment is dependent on identification and delineation of the area of early infarction on acute and subacute CTs. As this region may not be clearly visible on baseline NCCT within a few hours of stroke onset, at least some known examples of measuring early NWU have relied on CT perfusion (CTP) images to visually guide manual delineation of core infarct. In some studies where CTP was not available, NWU was estimated by measuring density within regions-of-interest (ROIs) placed within Alberta Stroke Program Early CT Score (ASPECTS) regions exhibiting early hypoattenuation and matched regions in the contralateral hemisphere. Measurement of NWU on follow-up NCCT typically requires manually outlining the visible region of infarction and flipping this manual ROI to create a homologous normal region for density assessment. This approach is time-consuming, subject to variability, and makes studying edema in large cohorts with NWU, although attractive in theory, challenging to perform in practice.

Prediction of which stroke patients are at high risk for malignant edema (the most common cause of early death) prior to their deterioration—in order to select those who need life-saving surgery—is typically imprecise based on clinical and known imaging variables. Automated analysis of stroke imaging is a growing field but has heretofore focused on admission/ED CT scans to evaluate which patients need reperfusion therapies. However, no known software or algorithms are able to process the follow-up imaging that stroke patients receive (at 24-hours) for important metrics and provide a customized prediction.

This Background section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### BRIEF DESCRIPTION

One aspect of this disclosure is a computer implemented method. The method includes receiving computed tomography (CT) scans of a patient after occurrence of a stroke in the patient, determining at least one cerebrospinal fluid (CSF) metric from the CT scans, and determining a likelihood of a future malignant edema occurring in the patient based at least in part on the at least one CSF metric.

Another aspect of this disclosure is a system for determining a likelihood of future malignant edema occurring in a patient. The system includes an input, a processor coupled to the input, and a memory coupled to the processor. The memory includes instructions that program the processor to receive, through the input, computed tomography (CT) scans of the patient after occurrence of a stroke in the patient, determine at least one cerebrospinal fluid (CSF) metric from the CT scans, and determine a likelihood of a future malignant edema occurring in the patient based at least in part on the at least one CSF metric.

Various refinements exist of the features noted in relation to the above-mentioned aspect. Further features may also be incorporated in the above-mentioned aspect as well. These refinements and additional features may exist individually or in any combination. For instance, various features discussed below in relation to any of the illustrated embodiments may be incorporated into the above-described aspect, alone or in any combination

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a simplified block diagram of an example imaging system.

FIG. 2 is a block diagram of an example server system for use in the imaging system of FIG. 1.

FIG. 3 is a block diagram of an example computing device for use in the imaging system of FIG. 1.

FIG. 4 is a flow diagram of an example method for the evaluation of the risk of malignant edema in a patient after a stroke.

FIG. 5A is an example of images for quantitative volumetric assessment of cerebral edema using cerebrospinal fluid (CSF) based biomarkers for one patient from the patients baseline computed tomography (CT) scan.

FIG. 5B is an example of images for the same patient as shown in FIG. 5A from a follow-up CT at about 26 hours after onset of stroke in the patient.

FIG. 6A is a graph of the association of change in CSF (ΔCSF) and hemispheric CSF ratio measured on follow-up CT scans after stroke onset for a group of patients.

FIG. 6B is a histogram of ΔCSF for the same group of patients as FIG. 6A.

FIG. 6C is a histogram of hemispheric CSF ratio for the same group of patients as FIG. 6A.

FIG. 6D is a histogram of transformed CSF ratio for the same group of patients as FIG. 6A.

FIG. 6E is a histogram of midline shift for the same group of patients as FIG. 6A.

FIG. 7A is a distribution of ΔCSF among a group of patients.

FIG. 7B is a distribution of CSF ratio among the same group of patients as in FIG. 7A.

FIG. 8 is an output of image analysis according to this disclosure for a follow-up CT scan at 72 hours after stroke for a patient with large right hemispheric infarction.

FIG. 9A is a graph of the correlation of hemispheric CSF ratio and relative hemispheric brain volume on 24-hour CT scans.

FIG. 9B is a graph of the correlation of hemispheric CSF ratio and midline shift on 24-hour CT scans.

FIG. 10 is a correlation matrix between various biomarkers of edema.

Similar reference symbols in the various drawings indicate like elements.

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION

Example embodiments of this disclosure include an automated imaging algorithm that can extract novel metrics from images of the brain of a patient who suffered a stroke. In some embodiments, the images are CT (computed tomography) scans of patients with stroke, and more specifically the total and hemispheric cerebrospinal fluid (CSF) volumes. These metrics are then input to a deep learning neural network prediction model to predict patients at risk for death or who would need surgery (combined with basic clinical features such as stroke severity). In some embodiments, an integrated software approach achieves this end-to-end—from entering imaging and clinical data—to processing the scan and providing a precision tailored prediction.

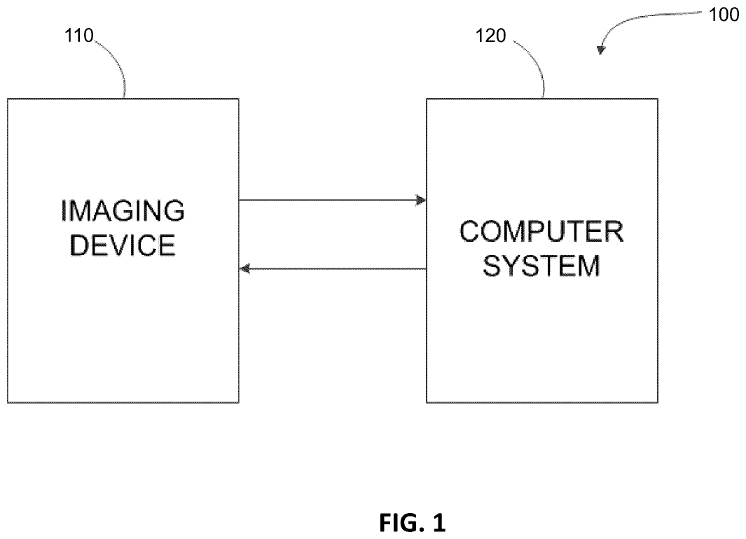

In various aspects, the methods described herein may be implemented using an imaging system. FIG. 1 is an illustration of an imaging system 100 in one aspect. As illustrated in FIG. 1, the imaging system 100 may include an imaging device 110 operatively coupled and/or in communication with a computer system 120. In the example embodiment, the imaging device 110 is a computed tomography (CT) system. Other embodiments may use any other suitable type of imaging device 110. In this aspect, the computer system 120 is configured to receive data including, but not limited to, a plurality of measured signals representing the imaged subject or object, sometimes referred to as image data, imaged data, or imaging data. The computer system 120 is further configured to execute a plurality of stored executable instructions encoding one or more aspects of the disclosed method as described herein above. In another aspect, the computer system 120 may be further configured to operate the imaging device 110 to obtain the plurality of measured signals representing the imaged subject or object that are received by the computer system 120, thereby enabling the disclosed methods by executing an additional plurality of stored executable instructions.

In some embodiments, the computer system 120 is located remote from the imaging system 100. The computer system may be communicatively connected to the imaging system 100 through a computer network, such as via a local area network, a wide area network, the Internet, and the like. In some embodiments, the imaging data from the imaging device 110 is uploaded to a remotely located server (such as by the computer system 120), from which the data may then be accessed by a remotely located computer. In other embodiments, the computer system 120 may itself be the remotely located server to which the imaging data is uploaded from the imaging device 110.

Although the disclosed systems and methods are described in connection with an imaging system 100, the disclosed systems and methods are operational with numerous other general purpose or special purpose imaging system environments or configurations. The imaging system environment is not intended to suggest any limitation as to the scope of use or functionality of any aspect of the disclosed systems and methods. Moreover, the imaging system environment should not be interpreted as having any dependency or requirement relating to any one or combination of components illustrated in the exemplary operating environment. Examples of well-known imaging systems, environments, and/or configurations that may be suitable for use with aspects of the invention include, but are not limited to, personal computers, server computers, hand-held or laptop devices, multiprocessor systems, microprocessor-based systems, set top boxes, programmable consumer electronics, mobile telephones, network PCs, minicomputers, mainframe computers, distributed computing environments that include any of the above systems or devices, and the like. By way of non-limiting example, embodiments of the invention are operational in a cloud computing environment such that the computer system 120 receives data from one or more computers (not shown) within the imaging system 100 or remote from the imaging system 100.

Computer systems, as described herein, refer to any known computing device and computer system. As described herein, all such computer systems include a processor and a memory. However, any processor in a computer system referred to herein may also refer to one or more processors wherein the processor may be in one computing device or a plurality of computing devices acting in parallel. Additionally, any memory in a computer device referred to herein may also refer to one or more memories wherein the memories may be in one computing device or a plurality of computing devices acting in parallel.

The term processor, as used herein, refers to central processing units, microprocessors, microcontrollers, reduced instruction set circuits (RISC), application specific integrated circuits (ASIC), logic circuits, and any other circuit or processor capable of executing the functions described herein. The above are examples only, and are thus not intended to limit in any way the definition and/or meaning of the term “processor.”

As used herein, the term “database” may refer to either a body of data, a relational database management system (RDBMS), or to both. As used herein, a database may include any collection of data including hierarchical databases, relational databases, flat file databases, object-relational databases, object oriented databases, and any other structured collection of records or data that is stored in a computer system. The above examples are example only, and thus are not intended to limit in any way the definition and/or meaning of the term database. Examples of RDBMS's include, but are not limited to including, Oracle® Database, MySQL, IBM® DB2, Microsoft® SQL Server, Sybase®, and PostgreSQL. However, any database may be used that enables the systems and methods described herein. (Oracle is a registered trademark of Oracle Corporation, Redwood Shores, California; IBM is a registered trademark of International Business Machines Corporation, Armonk, New York; Microsoft is a registered trademark of Microsoft Corporation, Redmond, Washington; and Sybase is a registered trademark of Sybase, Dublin, California.)

In one embodiment, a computer program is provided to enable the methods as described herein above, and this program is embodied on a computer readable medium. In an example embodiment, the computer system is executed on a single computer system, without requiring a connection to a server computer. In a further embodiment, the computer system is run in a Windows® environment (Windows is a registered trademark of Microsoft Corporation, Redmond, Washington). In yet another embodiment, the computer system is run on a mainframe environment and a UNIX® server environment (UNIX is a registered trademark of X/Open Company Limited located in Reading, Berkshire, United Kingdom). Alternatively, the computer system is run in any suitable operating system environment. The computer program is flexible and designed to run in various different environments without compromising any major functionality. In some embodiments, the computer system includes multiple components distributed among a plurality of computing devices. One or more components may be in the form of computer-executable instructions embodied in a computer-readable medium.

The computer systems and processes are not limited to the specific embodiments described herein. In addition, components of each computer system and each process can be practiced independent and separate from other components and processes described herein. Each component and process also can be used in combination with other assembly packages and processes.

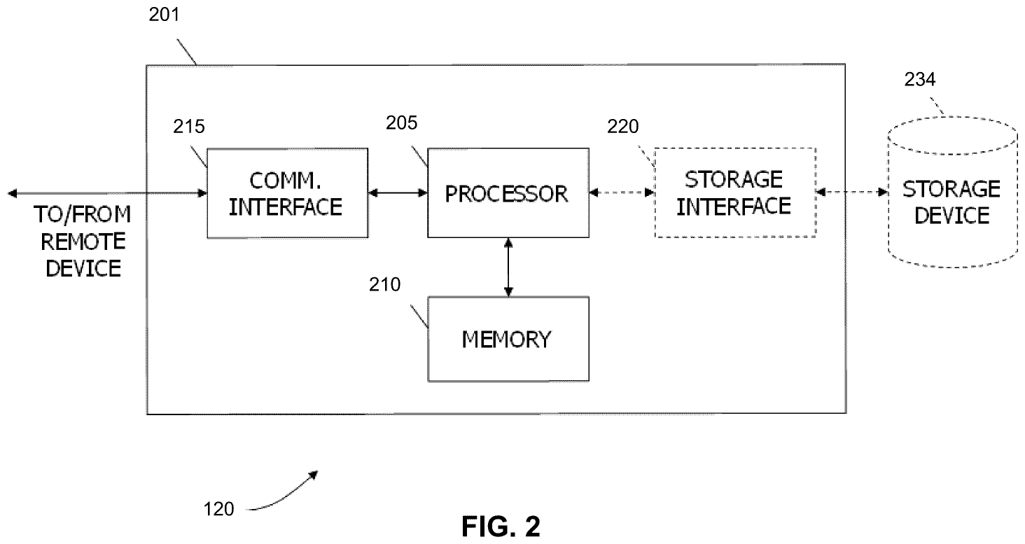

In one embodiment, the computer system 120 may be configured as a server system. FIG. 2 illustrates an example configuration of a server system 201 used to receive a reconstructed image or measured signals received from the subject or object to be reconstructed into an image representing the subject or object. Referring again to FIG. 2, server system 201 may also include, but is not limited to, a database server. In this example embodiment, server system 201 performs all of the steps used to implement the image reconstruction method as described herein above.

In this aspect, the server system 201 includes a processor 205 for executing instructions. Instructions may be stored in a memory area 210, for example. The processor 205 may include one or more processing units (e.g., in a multi-core configuration) for executing instructions. The instructions may be executed within a variety of different operating systems on the server system 201, such as UNIX, LINUX, Microsoft Windows®, etc. It should also be appreciated that upon initiation of a computer-based method, various instructions may be executed during initialization. Some operations may be required in order to perform one or more processes described herein, while other operations may be more general and/or specific to a particular programming language (e.g., C, C#, C++, Java, or any other suitable programming languages).

The processor 205 is operatively coupled to a communication interface 215 such that server system 201 is capable of communicating with a remote device, such as the imaging device 110 (see FIG. 1), a user system, or another server system 201. For example, communication interface 215 may receive requests (e.g., requests to provide an interactive user interface to receive sensor inputs and to control one or more devices of imaging system 100 from a client system via the Internet.

Processor 205 may also be operatively coupled to a storage device 234. Storage device 234 is any computer-operated hardware suitable for storing and/or retrieving data. In some embodiments, storage device 234 is integrated in server system 201. For example, server system 201 may include one or more hard disk drives as storage device 234. In other embodiments, storage device 234 is external to server system 201 and may be accessed by a plurality of server systems 201. For example, storage device 234 may include multiple storage units such as hard disks or solid state disks in a redundant array of inexpensive disks (RAID) configuration. Storage device 234 may include a storage area network (SAN) and/or a network attached storage (NAS) system.

In some embodiments, processor 205 is operatively coupled to storage device 234 via a storage interface 220. Storage interface 220 is any component capable of providing processor 205 with access to storage device 234. Storage interface 220 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 205 with access to storage device 234.

Memory area 210 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), non-volatile RAM (NVRAM), registers, hard disk memory, a removable disk, a CD-ROM, or any other form of computer-readable storage medium known in the art. The above memory types are exemplary only, and are thus not limiting as to the types of memory usable for storage of a computer program.

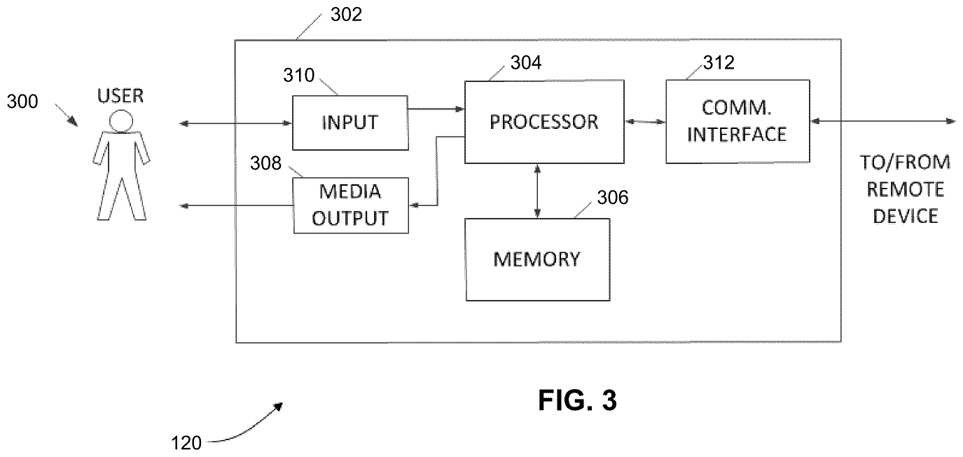

In another embodiment, the computer system 120 may be provided in the form of a computing device, such as a computing device 120 as illustrated in FIG. 3. Computing device 120 includes a processor 304 for executing instructions. In some embodiments, executable instructions are stored in a memory area 306. Processor 304 may include one or more processing units (e.g., in a multi-core configuration). Memory area 306 is any device allowing information such as executable instructions and/or other data to be stored and retrieved. Memory area 306 may include one or more computer-readable media.

In another embodiment, the memory included in the computing device 120 may include a plurality of modules (not shown). Each module may include instructions configured to execute using at least one processor 304. The instructions contained in the plurality of modules may implement at least part of the disclosed image reconstruction method by regulating a plurality of process parameters as described herein when executed by the one or more processors 304 of the computing device. Non-limiting examples of modules stored in the memory 306 of the computing device 120 include: a first module to receive measurements from one or more sensors and a second module to control one or more devices of the imaging system 100.

Computing device 120 also includes one media output component 308 for presenting information to a user 300. Media output component 308 is any component capable of conveying information to user 300. In some embodiments, media output component 308 includes an output adapter such as a video adapter and/or an audio adapter. An output adapter is operatively coupled to processor 304 and is further configured to be operatively coupled to an output device such as a display device (e.g., a liquid crystal display (LCD), organic light emitting diode (OLED) display, cathode ray tube (CRT), or “electronic ink” display) or an audio output device (e.g., a speaker or headphones).

In some embodiments, client computing device 120 includes an input device 310 for receiving input from user 300. Input device 310 may include, for example, a keyboard, a pointing device, a mouse, a stylus, a touch sensitive panel (e.g., a touch pad or a touch screen), a camera, a gyroscope, an accelerometer, a position detector, and/or an audio input device. A single component such as a touch screen may function as both an output device of media output component 308 and input device 310.

Computing device 120 may also include a communication interface 312, which is configured to communicatively couple to a remote device such as server system 201 or a web server. Communication interface 312 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory 306 is, for example, computer-readable instructions for providing a user interface to user 300 via media output component 308 and, optionally, receiving and processing input from input device 310. A user interface may include, among other possibilities, a web browser and an application. Web browsers enable users 300 to display and interact with media and other information typically embedded on a web page or a website from a web server. An application allows users 300 to interact with a server application.

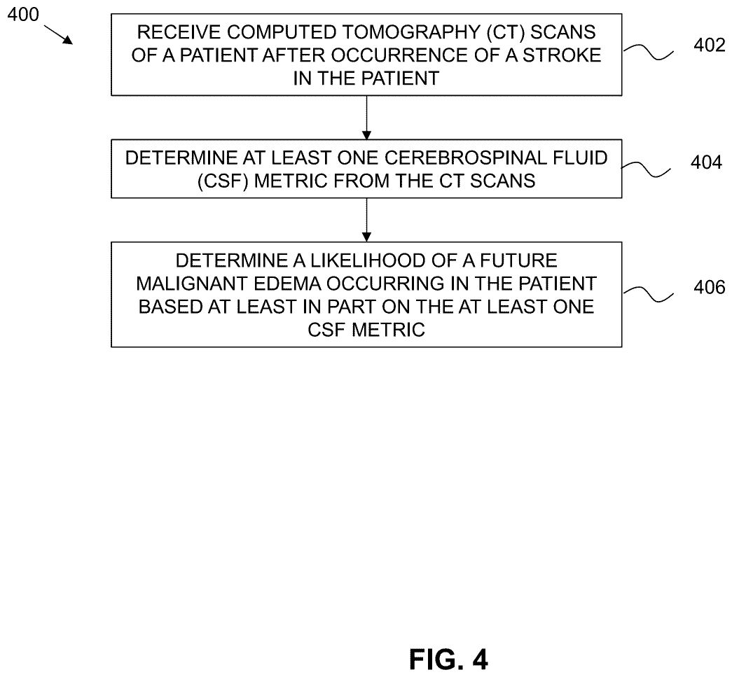

FIG. 4 is an example method 400 for the evaluation of the risk of malignant edema in a patient after a stroke. The method 400 may be performed by the computer system 120, by a remote computer communicatively coupled to the computer system 120, or by any other suitable computer system.

At 402, computed tomography (CT) scans of a patient after occurrence of a stroke in the patient are received. The CT scans (also sometimes referred to as image data or CT images) may be received directly from the CT scanner or may be retrieved from a server, database, or the like. The image data may be raw data from the CT scanner, or may be CT images reconstructed from the raw data. In some embodiments, the method 400 includes reconstructing the CT images from the raw data of the CT scans. As used herein, all slices of a CT scan are referred to collectively as a CT scan, a CT image, CT image data, or the like unless otherwise specified. In an example embodiment, the at least one CT scan of the subject includes a scan performed within twenty-four hours of the stroke. In some embodiments, the at least one CT scan of the subject additionally includes a baseline scan performed close to the onset of stroke in the patient (e.g., on admission to the hospital, within 6 hours of the onset of stroke, or the like), one or more scans performed more than twenty-four hours after the onset of stroke in the patient, or both.

At least one cerebrospinal fluid (CSF) metric is determined from the CT scans at 404. In some embodiments, the at least one CSF metric is a CSF ratio (sometimes referred to as a CSF hemisphere ratio or a hemispheric CSF ratio). The CSF ratio is a ratio of the volume of CSF in the stroke affected hemisphere of the patient's brain to the volume of the CSF in the contralateral hemisphere of the patient's brain. In embodiments in which more than one CT scan of the patient are received (with each scan being a scan performed at a different time), the CSF ratio may be calculated for each CT scan. In some embodiments, the CSF metric is a change in the CSF (ΔCSF) in the brain over time. In such embodiments, at least two CT scans from two different times are received. The CSF volume in the brain is calculated for both times, and the ΔCSF is calculated as the difference between the CSF volume of the later scan from the earlier scan. In at least some such embodiments, the earlier scan is the baseline scan performed within close to the onset of stroke in the patient.

In various embodiments, the method 400 includes, either as a separate step before 404 or as part of determining the CSF metric at 404, imaging analysis performed on the CT scans imaging data. Specifically, the received CT scans were processed (e.g., by the computer system 120), using any suitable algorithm, to extract the patient's brain from the CT images. Each extracted brain image is then registered to a CT-specific atlas template. For multiple, time-separated CT scans, each extracted brain image is also co-registered to the earliest (e.g., the baseline scan or other scan closest in time to the onset of stroke) of the CT images. This co-registration may aid in maintaining consistency of cranial volumes. In some embodiments, the system calculates the cranial volume of the patient as an additional metric. CSF segmentation is performed to identify the CSF in the brain for each registered brain image. In some embodiments, the CSF in the ventricles, sulci, and cisterns are segmented from each registered brain image using a machine learning algorithm, such as a deep learning algorithm pre-trained on appropriate training data. The CSF for each hemisphere of the brain is identified in each CT scan. In some embodiments, to determine the CSF in each hemisphere, the geometric midline of the atlas image was translated onto each slice of each CT image as the midline of the brain image. However, the CSF for the brain and each hemisphere is determined, the computer system 120 determines the total volume of CSF in the brain for each CT scan and/or determines the volume of CSF in each hemisphere (as defined by the translated midline) for each CT scan. When multiple time separated CT scans are included, the system may determine the ΔCSF as the total volume of CSF from the later in time CT scan minus the total volume of CSF from the earlier in time CT scan. The system determines the CSF ratio by dividing the volume of CSF in the ipsilateral hemisphere (the stroke affected hemisphere) by the volume of CSF in the contralateral hemisphere.

At 406, a likelihood of a future malignant edema occurring in the patient is determined based at least in part on the at least one CSF metric. In the example embodiment, the likelihood is determined by inputting the at least one CSF metric into a prediction algorithm, either alone or with other metrics included. In the example embodiment, the prediction algorithm is a recurrent neural network that employs a long short-term memory (LSTM) architecture (referred to sometimes herein as an LSTM neural network or an LSTM network). The inputs to the trained LSTM neural network include the cranial volume, the CSF volume, and the CSF ratio. The LSTM neural network outputs a percentage likelihood of developing a future malignant edema. In some embodiments, a midline shift and an infarct volume for the patient are also input to the LSTM neural network (which has been trained for use with such additional metrics). In still other embodiments, a regression model is employed on the at least CSF metric (alone or with other metrics) to determine a likelihood of a future malignant edema occurring. In still other embodiments, any other suitable machine learning algorithm may be used to determine the likelihood of a future malignant edema occurring.

In some embodiments, the likelihood is determined by comparison of the CSF metric to one or more thresholds. For example, a CSF ratio greater than a first threshold may be determined to have a low likelihood of a future malignant edema, a CSF ratio less than a second threshold may be determined to have a high likelihood of a future malignant edema, and a CSF ratio between the two thresholds may be determined to have a moderate likelihood of a future malignant edema. Of course, any number of thresholds may be used to increase or decrease the granularity of the determinations. Further, each threshold may be associated with a percentage (e.g., a 10% chance of developing malignant edema) rather than a relative likelihood.

In some embodiments, the method 400 includes displaying the determined likelihood of a future malignant edema occurring in the patient on a display device. In some embodiments, this includes displaying the at least one CSF metric used in the determination and/or one or more of the CT images. In some embodiments, the system may determine a recommended action to be taken based on the determined likelihood of a future malignant edema and may display such a recommendation to the user.

Determinations of the likelihood of a future malignant edema occurring in a patient made in accordance with the teachings of this disclosure may allow for earlier identification and more accurate identification of patients at risk of a future malignant edema. This may, in turn, allow for earlier intervention and treatment of at-risk patients, which may improve the likelihood of a positive outcome for the patient.

Additional details and features will be described in the examples described below.

### Example 1

Overview Cerebral edema has primarily been studied using midline shift or clinical deterioration as endpoints which only capture the delayed/severe manifestations of a process affecting many stroke patients. Quantitative imaging biomarkers that measure edema severity across the entire spectrum could improve its detection as well as identify relevant mediators of this important stroke complication.

An automated image analysis pipeline was used in this example to measure displacement of CSF (ΔCSF) and ratio of lesional versus contralateral hemispheric CSF volume (CSF ratio) in a cohort of 935 hemispheric stroke patients with follow-up CT scans median 26 hours (IQR 24-31) after stroke onset. Diagnostic thresholds were determined based on comparison to those without any visible edema and baseline clinical and radiographic variables were modeled against each edema biomarker and assessed how each biomarker was associated with stroke outcome (modified Rankin scale at 90 days).

ΔCSF and CSF ratio were correlated with midline shift (r=0.52 and −0.74, p<0.0001) but exhibited broader ranges. ΔCSF of greater than 14% or CSF Ratio below 0.90 identified those with visible edema: over half of the stroke patients met these criteria, compared with only 14% who had midline shift at 24 hours. Predictors of edema across all biomarkers included higher NIHSS, lower Alberta Stroke Program Early CT Score (ASPECTS) and lower baseline CSF volume. History of hypertension and diabetes (but not acute hyperglycemia) predicted greater ΔCSF but not midline shift. Both ΔCSF and lower CSF ratio were associated with worse outcome, adjusting for age, NIHSS, and ASPECTS (OR 1.7, 95% CI 1.3-2.2 per 21% ΔCSF).

Cerebral edema can be measured in a majority of stroke patients on follow-up CTs using volumetric biomarkers evaluating CSF shifts, including in many without visible midline shift. Edema formation is influenced by clinical and radiographic stroke severity but also chronic vascular risk factors and contributes to worse stroke outcomes.

Introduction Cerebral edema frequently develops in the hours to days after ischemic brain injury; the resultant increase in tissue water content leads to an increase in brain volume. Such brain swelling is a major cause of neurologic deterioration and death after ischemic stroke. Yet, there is increasing recognition that edema impacts not only those malignant cases, but that it may impair functional recovery in a much larger proportion of hemispheric (non-lacunar) stroke patients. Understanding the mediators of edema across all stroke patients is limited by a dearth of accessible, sensitive biomarkers that assess its severity across the full biologic spectrum. Prior studies have either relied on serial MRI analysis, not feasible for large pragmatic cohorts, or utilized midline shift, a crude measure of decompensated edema that is absent in the majority of those with clearly visible edema. Recently densitometric and volumetric edema biomarkers have been developed that may afford greater insights into the biologic mediators and impacts of edema after stroke.

Displacement of CSF, measurable between baseline and follow-up CT can serve as a quantitative biomarker of edema. This biomarker, ΔCSF, was correlated with eventual midline shift, but was measurable by 24-hours after stroke, when midline shift is generally absent to minimal. A second related biomarker, the ratio of volumes of CSF in each hemisphere (lesional vs. contralateral) indicates that reduced hemispheric CSF ratio on follow-up CT is strongly related to greater edema formation. In this example, both CSF-based biomarkers are applied to a larger cohort of stroke patients, using an integrated image analysis pipeline to facilitate volumetric assessment of edema severity. Assessing edema using this volumetric approach may highlight its broader incidence and facilitate a greater understanding of edema biology, opening up insights into key stroke mechanisms and targets.

Methods Study Participants—Patients enrolled in a prospective cohort study (the Genetics of Neurological Instability after Ischemic Stroke, GENISIS) at three sites between 2008 and 2018 were retrospectively evaluated for eligibility [14]. All participants presented within six hours of stroke onset. Those with at least one follow-up CT performed within 96 hours of stroke onset were selected. Those with unknown/unclear stroke onset time, if baseline CT already showed well-developed subacute infarction (suggesting time of stroke was likely earlier than indicated; acute stroke-related hypodensity was acceptable), if the stroke was located in the brainstem or cerebellum, or if the final discharge diagnosis was not stroke were excluded. All participants provided informed consent for their involvement in the prospective study, which included collection of clinical and imaging data. The STROBE observational cohort guidelines were followed.

Clinical Data—NIHSS scores were obtained at baseline and at 24-hours. Serum glucose and blood pressure were obtained on presentation. Study data including subject demographics, medical history and acute interventions were entered prospectively into a study-specific database. The majority of study participants were enrolled prior to the widespread adoption of multimodal imaging and thrombectomy so we did not have data on site of occlusion, collaterals, or perfusion deficits. TOAST stroke subtype was adjudicated by an experienced stroke physician based on testing data available at hospital discharge. Early clinical deterioration was defined as an increase in NIHSS from baseline to 24 hours. Malignant edema was defined for this example as midline shift associated with clinical deterioration (at any time), resulting in need for hemicraniectomy, osmotic therapy, or death. Functional outcome was determined by evaluating disability (using the modified Rankin Scale, mRS) at stroke follow-up visits or by telephone interview at 90-days.

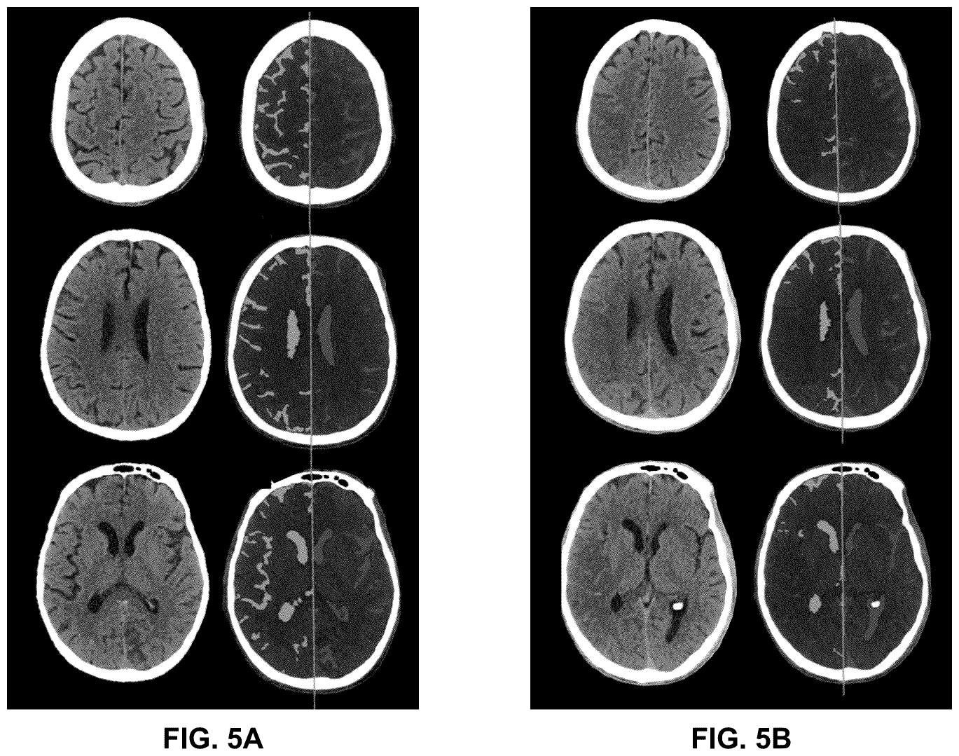

Imaging Data—All patients underwent CT imaging at baseline (within six hours of onset) and the majority had repeat CT close to 24-hours after presentation, especially those who received acute interventions like tPA; additional imaging was performed at the discretion of the treating team. The baseline and repeat CT scan closest to 24-hours were selected for analysis of early edema formation. These images were processed using a pipeline that automatically extracts quantitative metrics of CSF volume. This includes global and hemispheric CSF volumes at each time point; the hemispheric CSF ratio was calculated by dividing the CSF volume in the affected hemisphere (if stroke side was known, or side with lower volume if not) by the contralateral hemisphere CSF volume. FIGS. 5A and 5B are example images for quantitative volumetric assessment of cerebral edema using CSF-based biomarkers produced for one patient as described above. FIG. 5A includes the patients baseline CT and FIG. 5B is the follow-up CT at about 26 hours. In both, the original non-contrast CT is on the left side for three axial levels. The right side shows CSF from right (green) and left (red) hemispheres automatically segmented. The follow-up CT in this example shows a moderate sized right MCA stroke with some edema but without midline shift. ΔCSF was measured at 18% (baseline volume 198 ml, follow-up 163 ml). CSF ratio reduced from 0.98 to 0.73.

ΔCSF was calculated as the percent change in global CSF volume between baseline and follow-up CTs. Alberta Stroke Program Early CT Score (ASPECTS) was assessed manually on the baseline CT. ASPECTS was dichotomized into good and bad scores using a threshold of seven or less. Midline shift was assessed by the maximal displacement of the septum pellucidum on all follow-up CTs, with peak midline shift defined as greatest measured on all available CTs. Total volume of infarct-related hypodensity visible at 24-hours was measured using an automated segmentation algorithm, confirmed by manual review (REF). Hemorrhagic transformation was classified using an established grading system. Patients with PH-2 class of large parenchymal hematoma on follow-up CTs were excluded as the mass effect from these would confound any evaluation of midline shift and volumetric measures of edema.

Statistical Analysis—The distribution of ΔCSF was evaluated for outliers, identifying cases where CSF volume increased more than 20% (1.5 times the interquartile range above the lowest quartile) and manually reviewing segmentation results for technical errors. The range of both ΔCSF and CSF ratio measurements were studied to determine a threshold for defining presence of pathologic edema: to do this, a control group of those within the cohort without visible infarction, midline shift, or hemorrhage on follow-up CT (i.e. no edema) was selected. The first approach was to compute the 95% confidence interval of ΔCSF and CSF ratio values in that subgroup, using bootstrapping. The upper (for ΔCSF) and lower (for CSF ratio) limit of each biomarker was selected as one possible threshold. In addition, the package OptimalCutpoints was applied in R to compare distributions of biomarkers between these non-edema controls and the remainder of the population. This provides an optimal cut-point to distinguish those with visible edema from those without, optimizing sensitivity and specificity and provides the area-under-curve (AUC) for the receiver-operating-characteristic curve.

To satisfy the residual assumption for linear regression, the skewed CSF ratio data were first transformed using the cube root of the original ratio measurement subtracted from 1.0 (the maximal value); higher values representing more edema [23]. Linear regression models were applied using ΔCSF and transformed CSF ratio as outcomes. Midline shift was highly zero-inflated and so it was analyzed using a Tobit regression model (ref). Logistic regression was then performed using dichotomized mRS (0-2 was considered good recovery), to evaluate whether continuous edema measures were independently associated with outcome. OptimalCutpoints was applied to evaluate the AUC and thresholds at which each biomarker best predicted malignant edema and poor outcome.

In all models, explanatory variables were first evaluated using bivariate modeling; the Benjamini & Hochberg method was used to adjust for multiple testing in order to select those significantly associated with each outcome for inclusion in the multivariable models with p<0.15. Missing data was rare (<5%) and so was not imputed. The Chi-square test was used to compare fit of nested models and Aikake Information Criteria (AIC) values were used to compare non-nested models. Sensitivity analyses were performed limiting to those with NIHSS above 7 (a surrogate for larger strokes, where edema would be more prevalent), excluding those with lacunar strokes (based on TOAST classification) to focus on only larger strokes, and evaluating CSF ratio only in those with known stroke side (i.e. excluding those where stroke side was imputed based on lower volume). All statistical analyses were conducted using two-sided tests at a significance of 0.05 using R (R Foundation for Statistical Computing, Vienna, Austria).

Results Study Cohort—935 stroke patients with follow-up imaging were analyzed out of 1,356 enrolled at the three participating sites who had provided imaging. Thirty-six patients had large parenchymal hematoma on follow-up CT and were excluded as were four outliers, where technical issues resulted in artifactual CSF volumes on follow-up CT. Of those 899 included, median NIHSS was 8 (IQR 5-15) and 72% were treated with tPA. Median time to baseline CT (available in 886) was 1.8 hours (IQR 1.1-3.3). ASPECTS was a median of 10 (IQR 9-10) but 98 (11%) had scores of seven or less. The most common TOAST stroke etiology was cardioembolic (39%); less than 5% had lacunar strokes. Poor functional outcome (mRS 3-6) occurred in 337 (39%).

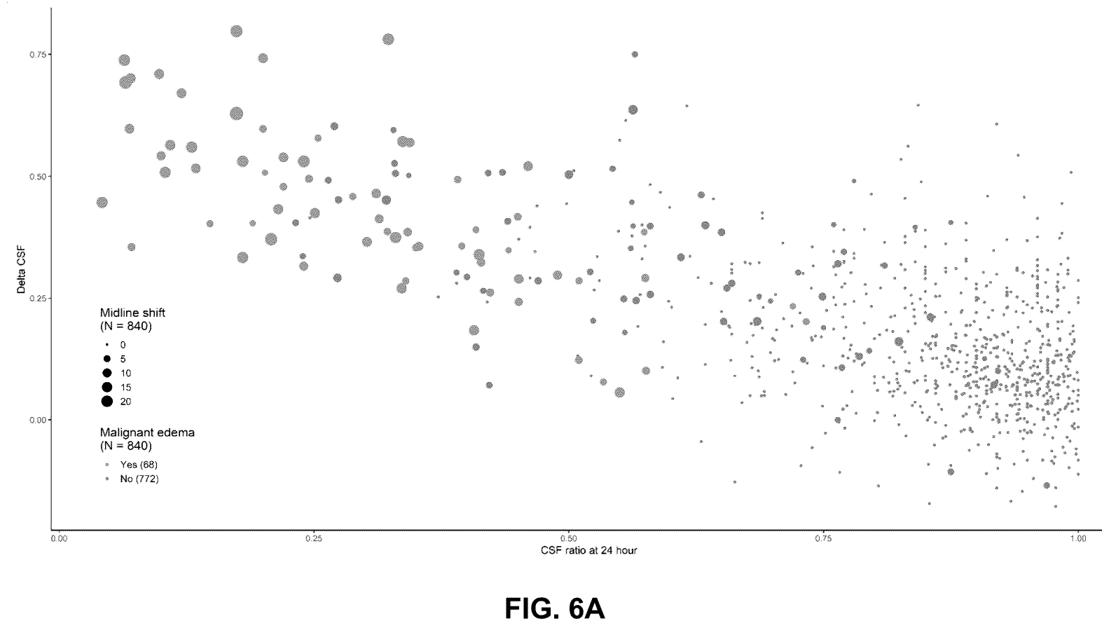

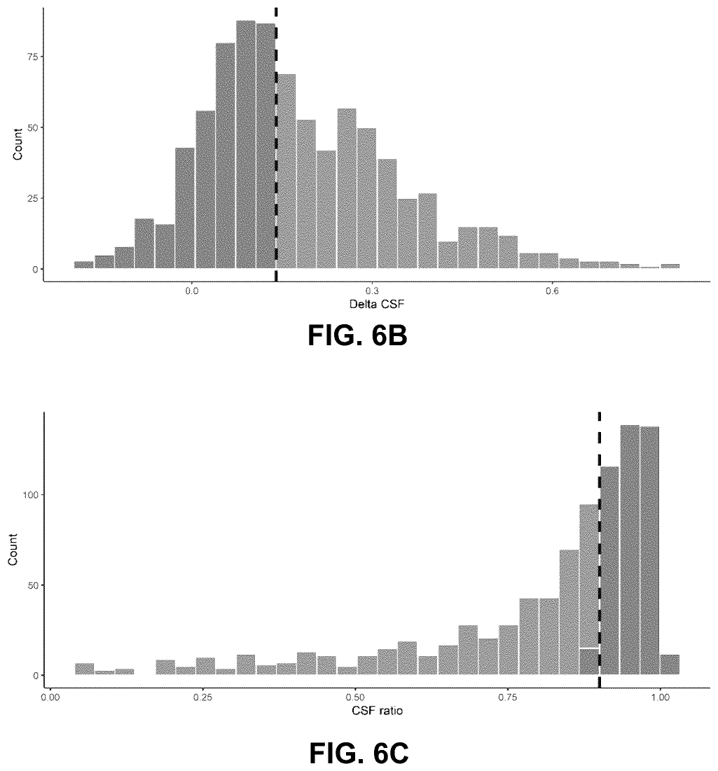

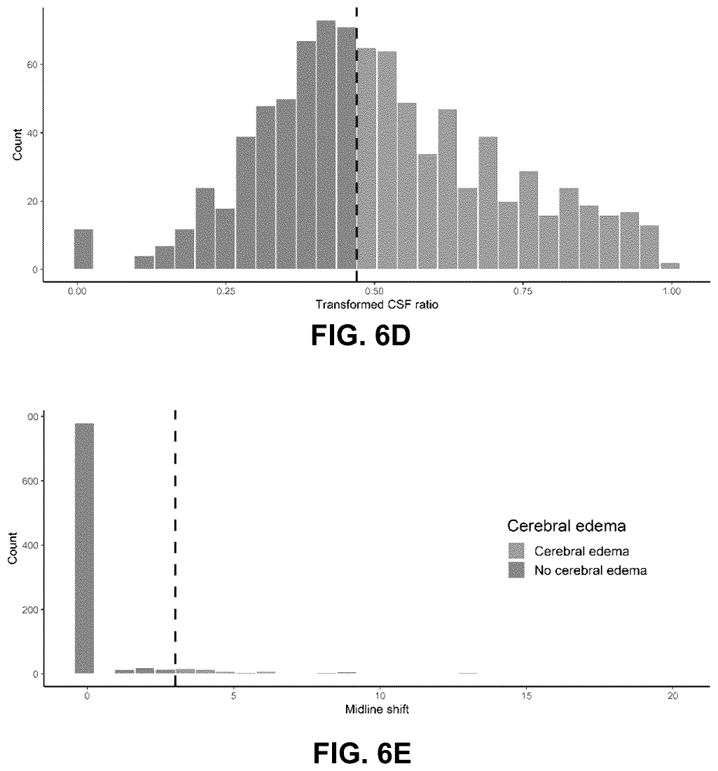

Incidence of Cerebral Edema on Follow-up Imaging—The median time to follow-up CT was 26 hours (IQR 23-31). The median ΔCSF was 15% (IQR 7-28) and median hemispheric CSF ratio was 0.89 (0.74-0.95) across the entire cohort. There was a strong correlation between larger ΔCSF and lower CSF ratio on follow-up CT (r=−0.63, p<0.0001), as shown in FIG. 6A. Both CSF ratio (r=−0.74) and ΔCSF (r=0.52) were also correlated with greater peak midline shift (both p<0.001) and ΔCSF was higher and CSF ratio markedly lower at 24-hours in those with malignant edema. In those without visible stroke or hemorrhage on follow-up CT (i.e. edema controls, n=167), the median ΔCSF was 9% (95% CI 7.7-10.9%) and median CSF ratio on follow-up CT was 0.94 (0.93-0.95). Applying thresholds based on the upper limit of these confidence intervals (11% for ΔCSF and 0.93 for CSF ratio) to define presence of measurable edema (i.e. beyond what is expected in stroke patients without any edema), 517 (61%) and 593 (66%) of the overall stroke cohort were found to have measurable edema at follow-up using ΔCSF and CSF ratio, while midline shift was only present in 126 (14%) at 24-hours and 155 (16.5%) at peak, with 93 (10%) having MLS≥3 mm. Using OptimalCutPoints, the optimal threshold to distinguish edema cases from controls was 14% for ΔCSF and 0.90 for CSF ratio. Applying this more conservative cut-point, we still found 441 (52%) and 483 (53%) had edema based on ΔCSF and CSF ratio measurements (distribution of these three edema biomarkers is shown in FIGS. 6B-6E, divided by those with vs. without edema based on these thresholds). FIG. 6A graphs the association of ΔCSF and hemispheric CSF ratio measured on follow-up CT scans at 12-96 hours (median 26 hours) after stroke onset in 840 patients. The size of the dots represent midline shift (see legend) and dots in red represent patients who developed malignant cerebral edema. FIG. 6B is a histogram of ΔCSF, FIG. 6C is a histogram of hemispheric CSF ratio, FIG. 6D is a histogram of transformed CSF ratio, and FIG. 6E is a histogram of midline shift, all of which highlight those in red who meet criteria for edema based on thresholds (ΔCSF>14%, CSF ratio<0.90, CSF ratio transformed>0.47, midline shift>3-mm).

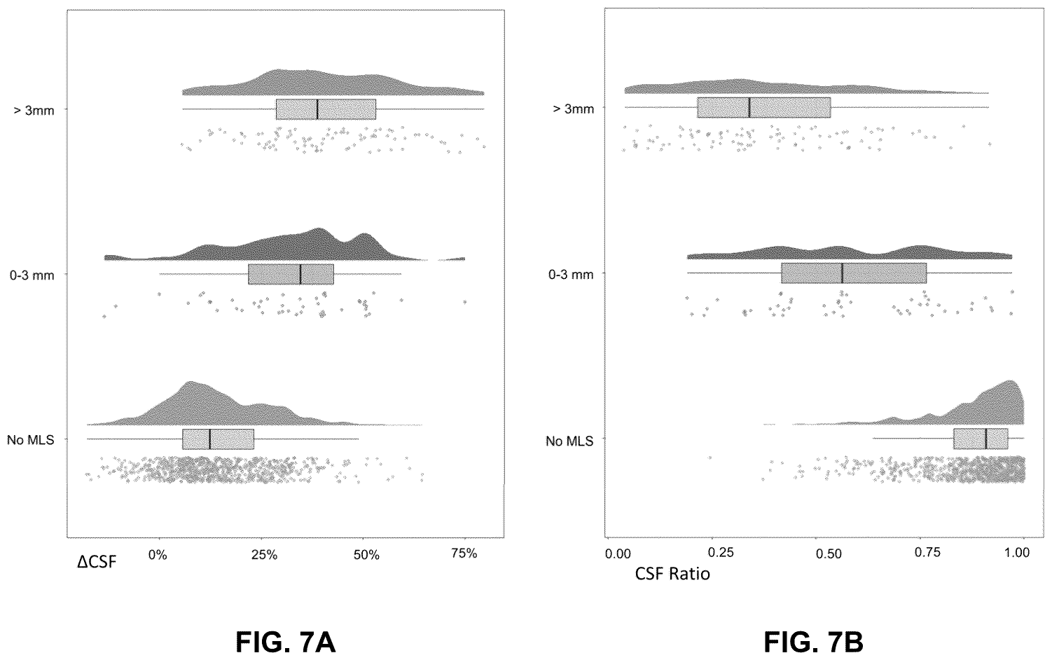

Furthermore, although ΔCSF and CSF ratio were greater in those with midline shift, as shown in FIGS. 7A and 7B, they still exhibited a broad dynamic range and demonstrated measurable edema even in those without any midline shift. FIG. 7A is a distribution of ΔCSF amongst those with midline shift (MLS) greater than 3 mm, with midline shift of 0-3 mm, and in those without midline shift. FIG. 7B is a distribution of CSF ratio amongst those with midline shift (MLS) greater than 3 mm, with midline shift of 0-3 mm, and in those without midline shift.

Variables Influencing Edema Formation—The impact of baseline variables on early edema severity, using CSF biomarkers and midline shift, measured at 24 hours was evaluated. There were significant univariate associations of higher NIHSS with more edema, using all three biomarkers. In addition, variables from baseline CT were consistently associated with greater severity of edema, including lower ASPECTS, lower CSF volume, and lower hemispheric CSF ratio. A history of diabetes and history of hypertension were associated with only greater edema using ΔCSF. Younger age was associated with all biomarkers while hyperglycemia was associated only with ΔCSF and midline shift, but non-significantly associated with CSF ratio. Higher blood pressure, gender, tPA treatment, and history of smoking or atrial fibrillation were not associated with any measures of edema severity. Time to baseline or follow-up CT, when added to these models, did not influence any relationships.

In multivariable models, higher NIHSS and lower ASPECTS remained associated with all measures of edema. Glucose was no longer associated with any edema outcomes after adjusting for NIHSS and ASPECTS. A history of diabetes and hypertension both remained significantly associated with greater ΔCSF but not lower CSF ratio or MLS, while baseline CSF volume and baseline CSF ratio were strongly associated with lower CSF ratio on follow-up CT. Baseline CSF volume was also associated with midline shift while age was no longer associated with any measures of edema after adjusting for CSF volume. These relationships were the same when restricting to subgroups with baseline NIHSS>7 or when excluding those with TOAST-defined lacunar strokes. Similarly, using CSF ratio only in those cases where stroke side was known (i.e. excluding those in which it was inferred based on lower hemispheric CSF volume without visible stroke) provided the identical modeling results.

Relationship of Edema Biomarkers with Clinical Deterioration—The outcome of malignant cerebral edema developed in 72 (8%). Those with malignant edema were younger, with higher NIHSS and admission glucose and lower ASPECTS. Both baseline CSF volume and hemispheric CSF ratio were also lower on baseline CT in those destined for malignant edema. The optimal cutoffs of these biomarkers for the outcome of malignant edema were 29% for ΔCSF (AUC of 0.885) and 0.58 for CSF ratio (AUC of 0.977). In comparison, evaluating midline shift provided AUC of 0.89 at an optimal threshold of 0.5 mm. Clinical deterioration (worsening of NIHSS at 24-hours) occurred in about 20%.

Impact of Edema on Stroke Outcome—Multivariable modeling found that age, higher NIHSS, tPA treatment, and lower ASPECTS were associated with worse outcome, but higher glucose and baseline CSF ratio were both no longer significantly associated (both p=0.07). When adding edema severity (each biomarker separately) to these outcome models, all edema measures were significantly associated with worse outcome and the models had better fit in comparison to models without edema severity (p<0.0001 for comparison). The optimal cut-points for poor outcome were ΔCSF above 15% and CSF ratio below 0.89. ΔCSF was associated with an odds ratio of 1.67 for poor outcome (95% CI 1.30-2.17, per 21% or one interquartile range), while lower CSF ratio had OR 2.33 (95% CI 1.75-3.13, per 0.21 lower ratio, or one IQR). The model incorporating CSF ratio had the lowest AIC of all biomarkers for stroke outcome. Results did not change when using CSF ratio only in cases where stroke side was known. Lower CSF ratio and greater midline shift were independently associated with worse outcomes even in those who did not have malignant edema (i.e. clinical deterioration), while ΔCSF was not.

Discussion In this example, automated assessment of edema severity was applied using two related CSF-based volumetric biomarkers in a cohort of almost one thousand hemispheric stroke patients. This allowed evaluation with greater sensitivity of how frequently edema can be observed after stroke and dissection more deeply of which variables influence all degrees of edema formation. Edema incidence using these biomarkers was compared to manually measured midline shift, the traditional metric of cerebral edema severity, as well as the categorical clinical outcome of malignant cerebral edema. Although both ΔCSF and the hemispheric CSF ratio were strongly correlated with midline shift and greater in those with malignant edema, they could be quantified more broadly across this large cohort, while midline shift and clinical deterioration occur later in the course and only capture those with more severe edema. Notably, less than ten percent developed malignant edema and less than twenty percent developed midline shift. In comparison, over 60% had significant ΔCSF (greater than 11%, the threshold based on normal variation in measurement seen in controls without any edema) and two-thirds had reductions in CSF ratio below 0.93 (i.e. 7% reduction in the stroke hemisphere relative to the contralateral hemisphere). Even using a more conservative threshold to distinguish those with edema (14% and 0.90), over half this stroke cohort exhibited measurable edema on early follow-up CT. There was a broad range of both biomarkers in those with and even in those without any midline shift, highlighting how measurable edema (i.e. accumulation of water around infarcted tissue, with brain swelling) is common in hemispheric stroke patients when assessed using sensitive biomarkers, as previously suggested in studies that required serial MRI.

Each edema biomarker was modeled to understand what baseline factors influence edema formation across its spectrum, and not just at its extremes. Many similarities were found in expected risk factors for edema, such as higher NIHSS and lower ASPECTS on baseline CT, across all measures of edema. CSF volume and CSF ratio on baseline CT also influenced measures of edema, while age did not, suggesting age is largely a surrogate for atrophy and intracranial reserve. However, certain variables were only discovered to influence edema severity when studied using these more sensitive biomarkers. For example, those with a history of diabetes and hypertension had greater ΔCSF, even adjusting for confounders such as glucose level, blood pressure on presentation, and NIHSS. This may suggest that chronic risk factors that cause microvascular changes in the cerebral circulation influence the severity of edema formation. Conversely, acute glucose level, which has been associated with edema in several studies that utilized categorical edema outcomes, was not associated with quantitative edema measures in this study, when adjusting for stroke severity and especially early ischemic changes (lower ASPECTS). It may be that hyperglycemia exacerbates very early ischemia, as reflected in lower ASPECTS, perhaps by impairing collateral circulation, and also shown by higher net water uptake in a recent study. The data of this example suggests that severity of volumetric brain swelling was not influenced by hyperglycemia, when adjusting for stroke severity, early edema and history of diabetes.

Finally, all quantitative and qualitative measures of edema were associated with worse stroke outcomes at 90-days. This was true not only in those with malignant edema (where severity of edema is clearly negatively associated with outcome) but also in those who did not develop clinical signs of edema. Further, the thresholds at which these biomarkers predicted worse outcome were quite similar to the cut-points extracted for presence of edema itself, suggesting that mild degrees of edema, without midline shift, are associated with poor stroke recovery. Incorporation of each edema metric on top of baseline prognostic variables (age, NIHSS, tPA, ASPECTS), significantly improved the model's performance. However, incorporating the CSF ratio on follow-up CT as an assessment of edema severity appeared to provide the best model fit. This aligns with expectations that lower CSF ratio was the strongest variable in a model that was able accurately predict malignant cerebral edema. Midline shift, especially when greater than 3-mm, has recently been shown to be an independent predictor of poor outcomes in hemispheric stroke. However, midline shift measurement requires subjective, manual assessment and is time-consuming to perform in large cohorts such as this one. The CSF measures of edema of this disclosure can both be extracted automatically, as they were from routine quality CTs from multiple sites in this example.

Conclusions Assessing edema using automatically-derived volumetric biomarkers that capture displacement of CSF on routine CT scans in large cohorts reveals how over half of hemispheric stroke patients develop measurable edema, compared with less than 20% using traditional measures. These more sensitive biomarkers can be applied to study factors that influence edema formation, here demonstrating that chronic hypertension and diabetes may contribute to greater edema severity. This approach also suggests that edema severity, across its entire spectrum and not just at the extremes, contributes negatively to stroke outcome, highlighting the importance of edema as a key mediator of secondary injury and impaired recovery for stroke patients. It suggests that studying edema as a continuous process has the potential to enhance our understanding of its biology and proposes that reductions in edema, for example by pharmacologic means, could represent an important avenue to improve stroke outcomes.

### Example 2

Summery Background and Purpose—Volumetric and densitometric biomarkers have been proposed to better quantify cerebral edema after stroke, but their relative performance has not been rigorously evaluated.

Methods—Patients with large vessel occlusion stroke from three institutions were analyzed. An automated pipeline extracted brain, CSF and infarct volumes from serial CTs. Several biomarkers were measured: change in global CSF volume from baseline (ΔCSF); ratio of CSF volumes between hemispheres (CSF ratio); and relative density of infarct region compared with mirrored contralateral region (net water uptake, NWU). These were compared to radiographic standards, midline shift and relative hemispheric volume (RHV) and malignant edema, defined as deterioration resulting in need for osmotic therapy, decompressive surgery, or death.

Results—255 patients with 210 baseline CTs, 255 24-hour CTs, and 81 72-hour CTs were analyzed. Of these, 35(14%) developed malignant edema and 63(27%) midline shift. CSF metrics could be calculated for 310 (92%) while NWU could only be obtained from 193 (57%). Peak midline shift was correlated with baseline CSF ratio (p=−0.22) and with CSF ratio and ΔCSF at 24 hours (p=−0.55/0.63) and 72 hours (p=−0.66/0.69), but not with NWU (p=0.15/0.25). Similarly, CSF ratio was correlated with RHV (p=−0.69/−0.78), NWU was not. Adjusting for age, NIHSS, and ASPECTS, CSF ratio (OR 1.92 per 0.1, 95% CI 1.52-2.52) and ΔCSF at 24 hours (OR 1.86 per 10%, CI 1.48-2.44) were associated with malignant edema.

Conclusion—CSF volumetric biomarkers can be automatically measured from almost all routine CTs and correlate better with standard edema endpoints than net water uptake.

Introduction Most patients with hemispheric strokes develop cerebral edema over the first few days after the ischemic insult. Cerebral edema is not only a major source of death and deterioration in the acute setting, but increased severity of edema has been independently related to worse long-term outcomes. Midline shift of variable degrees develops in almost half of all large vessel occlusion (LVO) strokes in the anterior circulation. While this may begin to be visible by 24 hours, it is primarily a manifestation of decompensated late-stage edema, peaking two to four days post-stroke. Detection of edema before midline shift and clinical deterioration occurs has been the subject of increasing interest. This would allow selection of patients for targeted interventions to reduce the impact of edema after stroke. Furthermore, quantification of edema across its full spectrum would facilitate a broader understanding of its biologic basis and contribution to stroke outcomes.

Prior approaches proposed to quantify edema have required advanced imaging, either comparison of DWI and FLAIR volumes on serial MRI examinations, or by measuring the relative hemispheric volume (RHV) on MRI, using the increase in size of the ipsilateral hemisphere as a surrogate of stroke-related swelling. Accurate automated CT-based measures would facilitate more widespread applications, as CT is the primary imaging modality after stroke. Infarct volume alone cannot be used to measure edema, as the infarct-related hypodensity comprises a variable combination of infarcted tissue and resulting edema. It is the excess water that contributes to hemispheric swelling and eventually results in midline shift. Several biomarkers that quantify water accumulation or hemispheric or global brain swelling have been developed over the past five years including net water uptake and those based on changes in CSF volume. Net water uptake (NWU) measures the relative density of the stroke lesion compared with a matching contralateral region as a surrogate for water accumulation. It has been applied primarily in the acute setting, where early edema formation on baseline CT has been related to worse collaterals, hyperglycemia, and to risk of malignant edema. Fewer studies have evaluated NWU as a marker of evolving edema on follow-up CTs; in fact, a recent evaluation of NWU challenged its validity in relation to reference standards of midline shift and RHV in the LVO population undergoing thrombectomy. Furthermore, measurement of NWU typically requires manual delineation of the infarct lesion, limiting ease of measurement. A second approach focuses on the volumetric assessment of swelling using the surrogate of global or hemispheric reductions in cerebrospinal fluid (CSF) volumes.

Both these densitometric and volumetric CT-based approaches have been proposed as means of better quantifying edema in order to predict midline shift and subsequent clinical deterioration (including the need for decompressive surgery) and as a therapeutic biomarker to assess response to emerging anti-edema interventions. Such tools could facilitate both clinical and research endeavors to mitigate the consequences of cerebral edema after stroke. However, it remains unclear how these two classes of biomarkers are related and whether one or the other better capture brain swelling and risk of deterioration, as no side-by-side studies have been performed. In this example, automated image analysis pipeline was used to measure both NWU and CSF volumes on serial CTs, the primary imaging modality used at most centers, to extract these biomarkers from a large multi-institutional cohort of anterior circulation LVO patients. Volumetric biomarkers such as global displacement of CSF (ΔCSF) and hemispheric CSF ratio were predicted to relate to established radiographic and clinical edema outcomes better than densitometric markers of edema like NWU.

Methods Cohort and Subject Selection—Clinical and imaging data were collected from three institutional stroke cohorts encompassing consecutive patients presenting over a period of about 40 months. The collection of data for each cohort was approved by the respective institution's human studies review board with a waiver of consent. Imaging data were collected in a central stroke repository. They were analyzed using an image analysis pipeline, described below. Subjects were included in this analysis if they were diagnosed with stroke due to an acute occlusion of the internal carotid or middle cerebral artery and had follow-up imaging performed in the first week after stroke. Time of last seen normal was used when exact stroke onset time was unknown. Follow-up CTs performed at least 12 hours after stroke onset and within one week were selected. The majority of patients had routine follow-up imaging performed at 24 hours after thrombolysis and/or thrombectomy per site protocol. A baseline CT was also available for most patients, allowing calculation of the change in CSF volume from baseline to each time point (ΔCSF). Clinical variables were abstracted from medical records (including structured stroke team notes) at each site by trained study investigators. These included demographics, last seen normal time, admission NIHSS, treatment with tPA and/or endovascular thrombectomy, reperfusion (using modified TICI grading, reported at the time of thrombectomy), and development of malignant cerebral edema, defined as radiographic evidence of brain swelling (i.e. midline shift) in association with both clinical deterioration and resulting in either death, surgical intervention, or treatment with osmotic drugs.

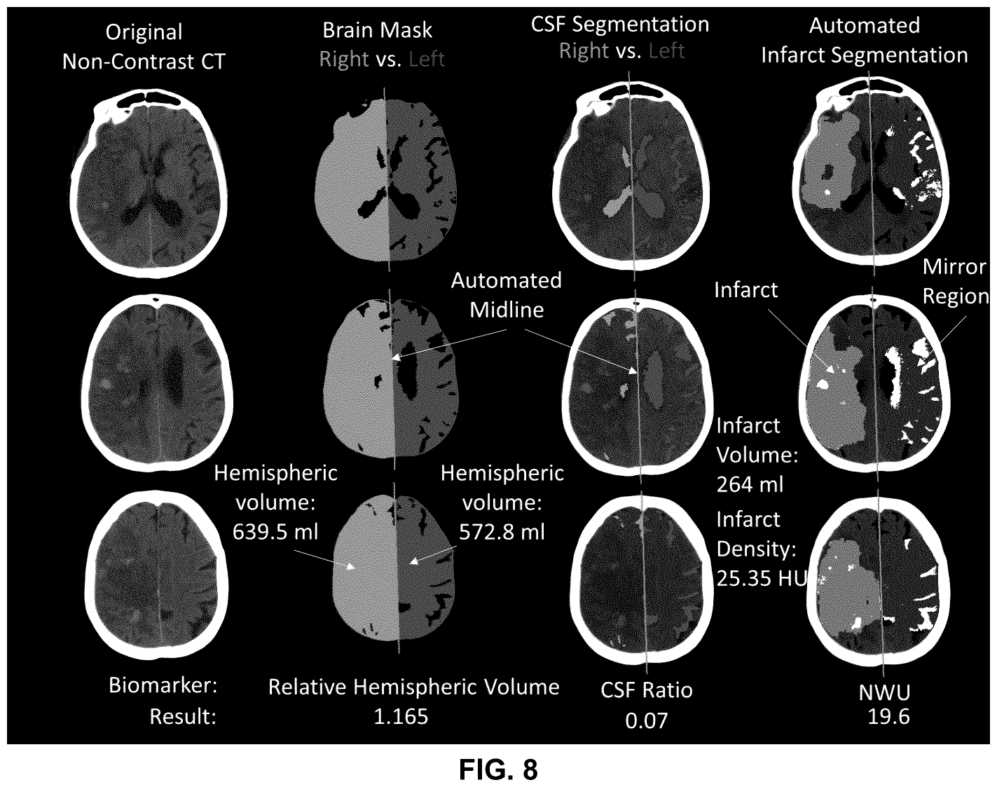

Imaging Analysis—A stroke edema pipeline was refined to extract multiple edema biomarkers. This included segmentation of CSF using a machine learning approach followed by extraction of the brain using BET (ref) and delineation of the two cerebral hemispheres by registering the brain midline to a standard CT atlas and then translating the midline on the atlas back to the patient's image. This allowed the separation of CSF and brain regions into hemispheres and the calculation of brain and CSF hemispheric ratios. The relative hemispheric volume (RHV) was the ratio of the volume of the brain in the stroke-affected hemisphere versus the contralateral hemisphere and the CSF ratio was the ratio of the volume of CSF in the stroke-affected hemisphere versus the contralateral hemisphere. NWU was automatically extracted from routine follow-up CTs by: 1) segmenting the hypodense region of acute cerebral infarction using a deep learning algorithm trained on 335 manually labeled examples; and 2) measuring the density of the infarct region and a mirrored region placed in the contralateral hemisphere, using the brain midline for reflection. Measurement of NWU was further refined by removing voxels of CSF from the infarct and mirror regions to avoid contamination, as well as thresholding the infarct region at 40 Hounsfield Units (HU) to remove high-density regions that could represent hemorrhagic transformation or contrast staining and would otherwise contaminate measurement. This pipeline was validated against manual measurement of NWU (both full-infarct and sampling-based methods). The final imaging results for an example patient are shown in FIG. 8. Specifically, FIG. 8 shows the output of image analysis for a follow-up CT at 72 hours after stroke for a patient with large right hemispheric infarction. The first column shows three axial slices from the non-contrast head CT (note: there are several regions of petechial hemorrhagic transformation and there was 3-mm of midline shift measured). The second column shows the extracted brain mask with automated delineation of the midline, separating the brain into right (ipsilateral) and left (contralateral) hemispheres. This allows calculation of the relative hemispheric volume (1.165). The third column shows the automated cerebrospinal fluid (CSF) segmentation, with hemispheric CSF separated using the same midline. The ratio of hemispheric CSF volumes is 0.07. In the final column, the orange region highlights the automated infarct segmentation. White regions within this represent areas thresholded out (i.e. Hounsfield units above 40). The purple region is the mirror of the infarct region within the contralateral hemisphere. The white regions within this represent areas of CSF not included in the mean density calculation. The ratio of infarct to mirror region densities is the net water uptake.

This pipeline was applied to calculate ΔCSF, the hemispheric CSF ratio, RHV, and NWU from this multi-institutional cohort of anterior circulation LVO patients. It was applied to both baseline and follow-up CTs, though those without visible infarction were excluded from NWU but not CSF calculation, as NWU cannot be calculated in the absence of a visible lesion (no infarct was present in 45% of scans at 24 hours and 21% at 72 hours; NWU could be measured from all but eleven of those with infarcts, representing a total of only 193 follow-up CTs or 57%). Manual review was performed of the automated imaging results for quality control and exclusion of: 1) cases where CSF segmentation or midline delineation failed (5 baseline, 15 24-hour CTs, and 9 72-hour CTs failed segmentation, ratio could not be obtained from one additional baseline and two scans at 24 hours—failed in 6% of 546 CTs); 2) cases without visible infarct or where automated infarct segmentation failed to capture the majority of the lesion. Midline shift was measured manually as the displacement of the midpoint of the septum pellucidum at the level of its maximal displacement and presence/severity of hemorrhagic transformation was ascertained.

Statistical Analysis—The first CT performed within 12 hours of stroke onset was considered the baseline. This was not available in 45 cases, mostly because it was performed at another facility or in cases of delayed presentation (when first CT was beyond 12 hours). Follow-up CTs were categorized into those closest to 24-hours (12-36 hours) and those closest to 72 hours (36-168 hours). The change in CSF volume between the baseline and each follow-up time point was calculated (ΔCSF). The peak midline shift was the maximal measured shift on all follow-up CTs. Correlations of biomarkers were performed using Spearman rho (p) given their non-parametric distribution. Partial correlations, adjusting for infarct volume, were also performed. Logistic regression was used to evaluate the independent association of each biomarker with malignant edema, adjusting for baseline variables, as well as tPA treatment, reperfusion status (in those undergoing thrombectomy). Given the a priori concern that the performance of NWU may differ in those undergoing thrombectomy and in those with hemorrhagic transformation, we performed sensitivity analyses stratified by thrombectomy and hemorrhagic transformation status. All analyses were performed in R (version 4.0.3, R Foundation for Statistical Computing, Vienna, Austria).

Results Subjects and Clinical Characteristics—Of 392 stroke patients evaluated, forty-seven were excluded for unknown stroke onset time, no LVO present on review of CT angiography, or occlusion not in the ICA or MCA vessels. No imaging was available for seven of the remaining 352 anterior circulation LVO subjects, and an additional eleven had no usable images after excluding poor quality or technically unusable scans. Among the remaining 334, 79 had only baseline CTs or no follow-up CTs beyond 12 hours, leaving 255 subjects with required follow-up imaging available.

The mean age in this cohort was 69 (SD 15) years, 118 (46%) were female, and 59 (23%) were non-white or Hispanic, by self-report. Baseline NIHSS was 15 (IQR 10-21) and glucose on presentation was 121 mg/dl (IQR 109-144). Forty patients had an occlusion of the internal carotid artery, in 129 it involved the proximal MCA, and in 84 the M2 or M3. Thrombolytic therapy was given in 110 (43%) and endovascular thrombectomy was attempted in 159 patients (62%). Successful reperfusion was recorded in 128 (80%). Malignant edema developed in 35 patients (12%) and PH-1 or PH-2 hemorrhagic transformation in 35 (14%). A comparison of the cohort demographics and clinical features divided by malignant edema status is shown in Table 1 below. In table 1, the following abbreviations are used: ASPECTS, Alberta Stroke Program Early CT Score; ICA, internal carotid artery; LVO, large vessel occlusion; M1, first segment of the middle cerebral artery; n, number; NIHSS, National Institutes of Health Stroke Scale, PH, parenchymal hematoma; tPA, tissue plasminogen activator; ΔCSF, change in CSF volume from baseline. Values in parenthesis represent percentages of each group (%), standard deviation (SD) when indicated, or interquartile range (IQR).

TABLE 1
Comparison of demographic, clinical, and imaging features in those
with versus without malignant edema in this study cohort

| | No Malignant | Malignant |
|---|---|---|
| | Edema | Edema |
| Feature | (n = 220) | (n = 35) |
| Age, years (SD) | 69 (14) | 67 (15) |
| Sex, female | 105 (48%) | 13 (37%) |
| Race, white non-Hispanic | 154 (70%) | 24 (69%) |
| NIHSS, baseline (IQR) | 14 (9-19) | 19 (15-23) |
| Glucose, mg/dl (IQR) | 120 (109-141) | 129 (109-160) |
| Systolic blood pressure, mm Hg | 150 (24) | 156 (18) |
| (SD) | | |
| ASPECTS (IQR) | 9 (8-10) | 7 (6-9) |
| LVO location, ICA or M1 | 140 (64%) | 29 (83%) |
| tPA given | 95 (43%) | 15 (43%) |
| Thrombectomy | 136 (62%) | 23 (66%) |
| Reperfusion 2b-3 | 115 (85%) | 13 (57%) |
| Hemorrhagic transformation (PH1 | 26 (12%) | 9 (26%) |
| or PH2) | | |
| Baseline relative hemispheric | 1.007 (0.02) | 1.014 (0.02) |
| volume (SD) | | |
| Baseline hemispheric CSF ratio | 0.95 (0.13) | 0.88 (0.13) |
| (SD) | | |
| Midline shift at 24-hours (IQR) | 0 (0-0) | 2.9 (0-6) |
| ΔCSF at 24-hours (%) (IQR) | 12% (5-27) | 49% (30-71) |
| Hemispheric CSF ratio at 24-hours | 0.82 (0.68-0.91) | 0.37 (0.30-0.60) |
| (IQR) | | |
| Net water uptake at 24-hours (%) | 21 (17-25) | 21 19-25) |
| (IQR) | | |

There were no differences in age, sex, racial identification, admission glucose or blood pressure between the groups. NIHSS was higher and ASPECTS was lower in those developing malignant edema. There was no difference in the rate of thrombolytic or endovascular treatment between the groups but successful reperfusion was significantly less likely in the malignant edema group. These patients were also more likely to have ICA or M1 segment occlusions.

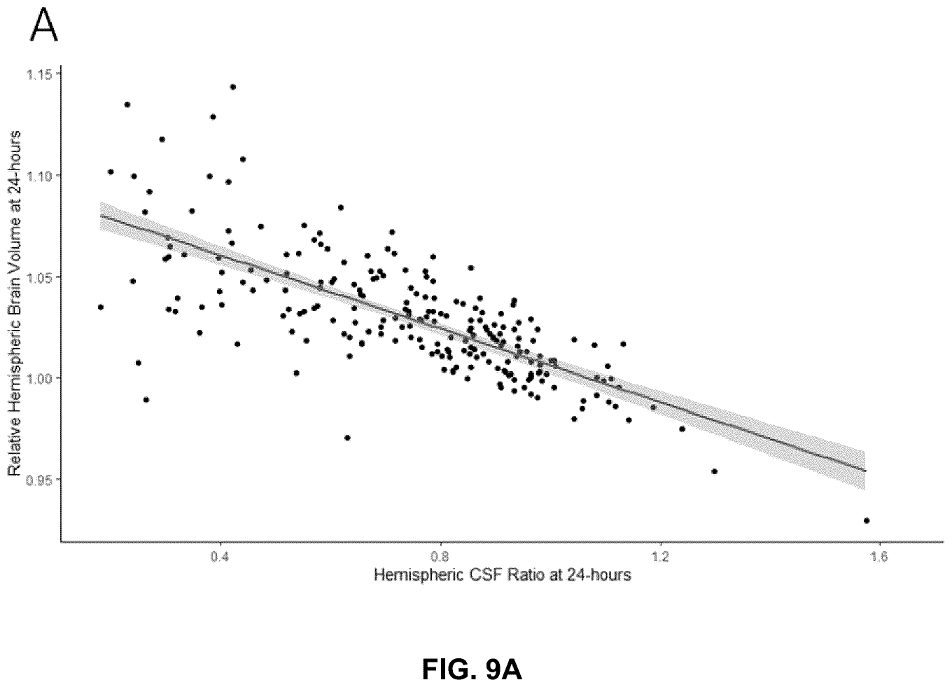

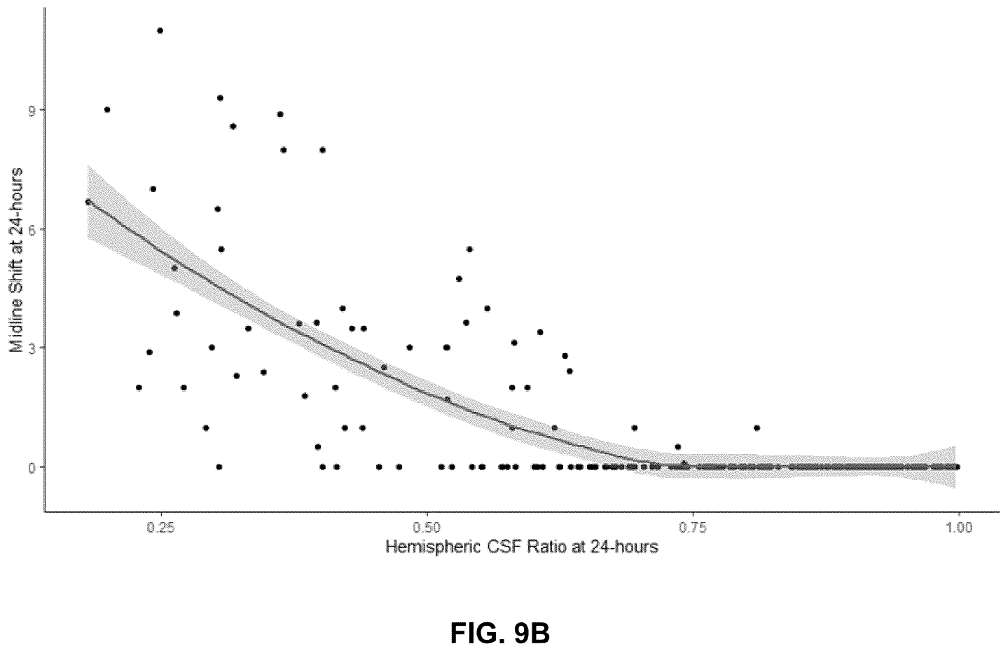

Measurement of Biomarkers—Scans were binned into baseline (n=210; median time from onset to CT 2.2 hours, IQR 0.9-4.3), 24-hour (n=255, median time 25.8 hours, IQR 20-31), or 72-hour (n=81, median time 70 hours, IQR 55-87). The values for each biomarker at each time point are shown in Table 2 below. Values in parentheses represent the interquartile range for each measurement. Midline shift was present in 55 patients (22%) at 24 hours and in 63 patients (27%) overall, although the median shift was still zero at both time points. Relative hemispheric volume (RHV) was significantly different from one on the baseline scan (i.e. two hemispheres were not equal in volume, p<0.001) but increased further at follow-up. The median reduction in CSF volume at 24 hours was 24 ml (IQR 8-49), representing a 14% change from baseline (IQR 6-30). In those with 72-hour scans, the ΔCSF was 34 ml (13-74) or 22% (9-41%). The CSF ratio was significantly below 1.0 at baseline (p<0.001) and was correlated with RHV at all three time points, as seen in FIG. 9A for 24-hour time point, p=0.74). FIG. 9A graphs the correlation of hemispheric CSF ratio and relative hemispheric brain volume on 24-hour CT scans (line represents linear regression with 95% confidence interval).

TABLE 2
Descriptive summary of edema biomarkers measured at
baseline and on follow-up CTs at 24- and 72-hours

| | Baseline | 24-hours | 72-hours |
|---|---|---|---|
| Number of scans analyzed | 210 | 255 | 81 |
| Time to scan (hours) | 1.9 (0.9-4.3) | 24.8 (19-29) | 71 (55-87) |
| CSF volume (ml) | 159 (122-202) | 127 (89-171) | 119 (86-159) |
| | (n = 205) | (n = 240) | (n = 72) |
| Midline shift (mm) | 0 (0-0) | 0 (0-0) | 0 (0-3.9) |
| Number with midline shift | 0 (0%) | 55 (22%) | 35 (43%) |
| Hemispheric CSF ratio | 0.95 (0.88-1.00) | 0.79 (0.59-0.92) | 0.65 (0.45-0.81) |
| | (n = 204) | (n = 238) | (n = 72) |
| Relative hemispheric | 1.008 (0.997-1.018) | 1.025 (1.01-1.05) | 1.04 (1.01-1.06) |
| volume | | | |
| Infarct hypodensity volume | N/A | 56 (22-145) | 103 (45-201) |
| (ml) | | (n = 140) | (n = 64) |
| Net Water Uptake (%) | 14.0 (8.0-19.3) | 21.2 (17.7-25.4) | 27.3 (23.2-32.3) |
| | (n = 9) | (n = 134) | (n = 59) |

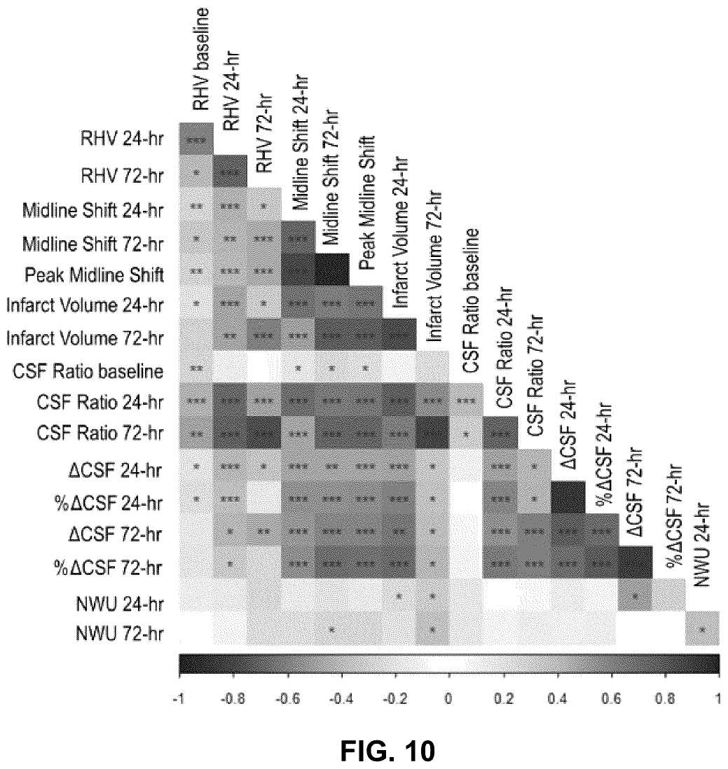

Correlations between Biomarkers— FIG. 10 shows the correlation matrix between the various biomarkers of edema. Peak midline shift was correlated most strongly with the CSF ratio at both 24- and 72 hours (rho=0.65 and 0.69) but also with ΔCSF. In contrast, there was only weak correlation between peak MLS and NWU at 24 hours (p=0.14, p=0.1) and at 72 hours (p=0.2′7, p=0.042). Evaluating the relationship of decreasing CSF ratio with midline shift suggested that when the ratio fell below 0.50 there was a sharp rise in midline shift as seen in FIG. 9B, which plots hemispheric CSF ratio and midline shift on 24-hour CT (line represents quadratic regression with 95% confidence interval), highlighting the strong associations of CSF volume-based metrics with midline shift and RHV. Even the CSF ratio on baseline CT was weakly correlated with peak midline shift (p=0.23, p=0.001) while baseline RHV was not. The association of CSF ratio and ΔCSF at 24 hours with midline shift remained significant even after adjusting for infarct volume (partial correlation 0.48, p<0.0001) as it did for ΔCSF (−0.35, =0.0001).

Association of Biomarkers with Malignant Edema—The hemispheric CSF ratio was lower in those destined to develop malignant edema even on baseline CT (0.88 vs. 0.95, p=0.02) while RHV was not (1.014 vs. 1.007, p=0.09). There were not enough baseline NWU (nine of which two had edema) measurements to compare it between edema groups. The total displacement of CSF (ΔCSF) was significantly greater (49% vs. 12%) and the CSF ratio significantly lower (0.37 vs. 0.83, both p<0.0001) at 24 hours, while NWU was similar (Table 1). Adjusting for age, NIHSS, tPA treatment, and ASPECTS, 24-hour CSF ratio was strongly associated with malignant edema (OR 1.95 per 0.1 decrease, 95% CI 1.52-2.59) while baseline CSF ratio was not (p=0.27). Notably, neither NIHSS nor ASPECTS were independently predictive of malignant edema after incorporating CSF ratio. Similarly, ΔCSF was independently associated with malignant edema (OR 1.87 per 10% decrease, 95% CI 1.47-2.49), while NWU was not (p=0.49). In fact, both CSF-based biomarkers had stronger associations than the reference standard, RHV. In those undergoing thrombectomy, successful reperfusion was associated with a lower risk of malignant edema (OR 0.21, 95% CI 0.07-0.60), adjusting for age, NIHSS, tPA, and ASPECTS. However, the CSF ratio at 24 hours remained significantly associated, even adjusting for reperfusion status (OR 1.89 per 0.1 decrease, 95% CI 1.41-2.67). Similarly, ΔCSF was associated with progression to malignant edema (OR 2.21 per 10%, 95% CI 1.53-3.65), while NWU was not. Finally, even excluding those who underwent thrombectomy, NWU was still not associated with edema, while CSF parameters were. Similarly, excluding the 38 patients with PH type of HT, there was still no correlation of NWU with midline shift or association with malignant edema.

Discussion In this analysis of 255 patients with stroke due to anterior circulation LVO, it was demonstrated that CSF-based volumetric biomarkers on serial head CTs correlate strongly with established imaging measures of edema and with the clinical outcome of malignant cerebral edema. A lower hemispheric CSF ratio at baseline and 24-hours was associated with greater midline shift and higher risk for malignant edema, as was a greater displacement of CSF (ΔCSF). Moreover, asymmetry of CSF between the two hemispheres (i.e. low CSF ratio) could be evaluated in all those with edema, even on admission, while midline shift was visualized in less than one quarter of patients at 24 hours. Evaluating CSF displacement offers an earlier imaging window into the evolution of edema than possible using midline shift or waiting till clinical deterioration occurs. In contrast, the densitometric biomarker, net water uptake, did not exhibit a relationship to any of these established measures. Furthermore, it was demonstrated that the volumetric biomarkers could be obtained from almost all CT scans while NWU could be measured in just over half, restricted to cases when a clear infarct was visible.

A comprehensive pipeline was employed to automatically extract both classes of biomarkers from serial routine head CTs obtained at three different institutions. Measurement of NWU typically requires manually outlining the region of infarction, slice-by-slice, and then mirroring this region to the contralateral hemisphere; an approach impractical for bedside assessment or for use in large cohort studies. This obstacle was surmounted by developing a NWU algorithm that automatically segments the hypodense infarct region and creates the mirror region.20 NWU assessment at follow-up also may be particularly susceptible to contamination from regions of hemorrhagic transformation or contrast within the infarct (for example, in those undergoing thrombectomy). In fact, at least one study of large hemispheric strokes demonstrated that NWU did not correlate with RHV (both manually measured), but did so weakly after excluding those with hemorrhage (p=0.21) or all those undergoing thrombectomy (p=0.45). The exclusion of such patients limits its broad applicability to study edema after a severe stroke. Our NWU method attempts to mitigate this by thresholding the region of infarction at an upper limit of 40 Hounsfield Units; while a slightly stronger correlation to midline shift and RHV was observed when excluding patients with hemorrhage or thrombectomy, it was still not significantly correlated with reference standards of edema. This suggests that NWU on follow-up CT does not capture the key aspects of brain swelling that are associated with deterioration. Instead, it may reflect different aspects of edema, perhaps the early ionic shifts that cause progressive tissue hypodensity but may not lead to as much brain swelling as the cytotoxic and vasogenic edema pathways that precipitate volumetric increases in the ipsilateral hemisphere and can lead to herniation. These are more strongly captured by measuring the displacement of hemispheric and global CSF. These volumetric biomarkers are more widely measurable at early time points (i.e. do not require a visible infarct or concurrent estimation of infarct from CT perfusion) and are strongly linked to clinically significant outcomes.

Machine Learning & Other Matters

The computer-implemented methods discussed herein may include additional, less, or alternate actions, including those discussed elsewhere herein. The methods may be implemented via one or more local or remote processors, transceivers, servers, and/or sensors, and/or via computer-executable instructions stored on non-transitory computer-readable media or medium.

Additionally, the computer systems discussed herein may include additional, less, or alternate functionality, including that discussed elsewhere herein. The computer systems discussed herein may include or be implemented via computer-executable instructions stored on non-transitory computer-readable media or medium.

In some embodiments, the design system is configured to implement machine learning, such that the neural network “learns” to analyze, organize, and/or process data without being explicitly programmed. Machine learning may be implemented through machine learning (ML) methods and algorithms. In an exemplary embodiment, a machine learning (ML) module is configured to implement ML methods and algorithms. In some embodiments, ML methods and algorithms are applied to data inputs and generate machine learning (ML) outputs. Data inputs may include but are not limited to: analog and digital signals, sensor data, image data, video data, patient data, and the like. ML outputs may include but are not limited to: digital signals, medical diagnoses, segmented images, health care predictions and guidance, and the like. In some embodiments, data inputs may include certain ML outputs.

In some embodiments, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, recurrent neural networks, Monte Carlo search trees, generative adversarial networks, dimensionality reduction, and support vector machines. In various embodiments, the implemented ML methods and algorithms are directed toward at least one of a plurality of categorizations of machine learning, such as supervised learning, unsupervised learning, and reinforcement learning.

In one embodiment, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function which maps outputs to inputs and utilize the predictive function to generate ML outputs based on data inputs. The example inputs and example outputs of the training data may include any of the data inputs or ML outputs described above. For example, a ML module may receive training data comprising data associated with different patients and their corresponding outcomes, generate a model which maps the patient data to the outcome data, and recognize potential future outcomes for patients.

In another embodiment, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship. In an exemplary embodiment, a ML module coupled to or in communication with the design system or integrated as a component of the design system receives unlabeled data, and the ML module employs an unsupervised learning method such as “clustering” to identify patterns and organize the unlabeled data into meaningful groups. The newly organized data may be used, for example, to extract further information about the potential classifications.

In yet another embodiment, ML methods and algorithms are directed toward reinforcement learning, which involves optimizing outputs based on feedback from a reward signal. Specifically ML methods and algorithms directed toward reinforcement learning may receive a user-defined reward signal definition, receive a data input, utilize a decision-making model to generate a ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In an exemplary embodiment, a ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict optimal constraints.

The computer-implemented methods discussed herein may include additional, less, or alternate actions, including those discussed elsewhere herein. The methods may be implemented via one or more local or remote processors, transceivers, servers, and/or sensors (such as processors, transceivers, and/or servers), and/or via computer-executable instructions stored on non-transitory computer-readable media or medium. Additionally, the computer systems discussed herein may include additional, less, or alternate functionality, including that discussed elsewhere herein. The computer systems discussed herein may include or be implemented via computer-executable instructions stored on non-transitory computer-readable media or medium.

As used herein, the term “non-transitory computer-readable media” is intended to be representative of any tangible computer-based device implemented in any method or technology for short-term and long-term storage of information, such as, computer-readable instructions, data structures, program modules and sub-modules, or other data in any device. Therefore, the methods described herein may be encoded as executable instructions embodied in a tangible, non-transitory, computer readable medium, including, without limitation, a storage device and/or a memory device. Such instructions, when executed by a processor, cause the processor to perform at least a portion of the methods described herein. Moreover, as used herein, the term “non-transitory computer-readable media” includes all tangible, computer-readable media, including, without limitation, non-transitory computer storage devices, including, without limitation, volatile and nonvolatile media, and removable and non-removable media such as a firmware, physical and virtual storage, CD-ROMs, DVDs, and any other digital source such as a network or the Internet, as well as yet to be developed digital means, with the sole exception being a transitory, propagating signal.

The patent claims at the end of this document are not intended to be construed under 35 U.S.C. § 112(f) unless traditional means-plus-function language is expressly recited, such as “means for” or “step for” language being expressly recited in the claim(s).

This written description uses examples to disclose various implementations, including the best mode, and also to enable any person skilled in the art to practice the various implementations, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A computer implemented method comprising:
receiving computed tomography (CT) scans of a patient after occurrence of a stroke in the patient;
determining at least one cerebrospinal fluid (CSF) metric from the CT scans; and
determining a likelihood of a future malignant edema occurring in the patient based at least in part on the at least one CSF metric.

2. The computer implemented method of claim 1, wherein determining at least one CSF metric comprises extracting an image of the patient's brain from the CT scans and identifying CSF in the extracted image of the patient's brain in the CT scans.

3. The computer implemented method of claim 2, wherein determining at least one CSF metric comprises determining a CSF ratio.

4. The computer implemented method of claim 3, wherein determining a CSF ratio comprises determining a midline of the extracted image of the patient's brain that defines a stroke affected hemisphere and a contralateral hemisphere, determining a volume of CSF in the stroke affected hemisphere and the volume of CSF in the contralateral hemisphere, and calculating the CSF ratio as the volume of CSF in the stroke affected hemisphere divided by the volume of CSF in the contralateral hemisphere.

5. The computer implemented method of claim 2, wherein the CT scans comprises a first CT scan and a second CT scan, the first CT scan being acquired at an earlier time than the second CT scan, extracting an image of the patient's brain from the CT scans comprises extracting a first image of the patient's brain from the first CT scan and extracting a second image of the patient's brain from the second CT scan, and identifying CSF in the extracted image of the patient's brain in the CT scans includes identifying a first volume of CSF from the first image of the patient's brain and a second volume of CSF from the second image of the patient's brain.

6. The computer implemented method of claim 5, wherein determining at least one CSF metric comprises determining a change in CSF (ΔCSF) in the patient's brain over time by subtracting the first volume of CSF from the second volume of CSF.

7. The computer implemented method of claim 2, wherein identifying CSF in the extracted image of the patient's brain comprises processing the extracted image of the patient's brain with a deep learning algorithm trained to perform CSF segmentation.

8. The computer implemented method of claim 1, wherein determining a likelihood of a future malignant edema occurring in the patient based at least in part on the at least one CSF metric comprises inputting the at least one CSF metric into a prediction algorithm.

9. The computer implemented method of claim 8, wherein inputting the at least one CSF metric into a prediction algorithm comprises inputting the at least one CSF metric into a recurrent neural network employing a long short-term memory (LSTM) architecture.

10. The computer implemented method of claim 1, wherein determining at least one CSF metric comprises determining a plurality of CSF metrics and determining a likelihood of a future malignant edema occurring in the patient based at least in part on the at least one CSF metric comprises determining a likelihood of a future malignant edema occurring in the patient based at least in part on the plurality of CSF metrics.

11. A system for determining a likelihood of future malignant edema occurring in a patient, the system comprising:
an input;
a processor coupled to the input; and
a memory coupled to the processor, the memory including instructions that program the processor to:
receive, through the input, computed tomography (CT) scans of the patient after occurrence of a stroke in the patient;
determine at least one cerebrospinal fluid (CSF) metric from the CT scans; and
determine a likelihood of a future malignant edema occurring in the patient based at least in part on the at least one CSF metric.

12. The system of claim 11, wherein the instructions program the processor to extract an image of the patient's brain from the CT scans and identifying CSF in the extracted image of the patient's brain in the CT scans.

13. The system of claim 12, wherein the at least one CSF metric comprises a CSF ratio.

14. The system of claim 13, wherein the instructions program the processor to determine the CSF ratio by determining a midline of the extracted image of the patient's brain that defines a stroke affected hemisphere and a contralateral hemisphere, determining a volume of CSF in the stroke affected hemisphere and the volume of CSF in the contralateral hemisphere, and calculating the CSF ratio as the volume of CSF in the stroke affected hemisphere divided by the volume of CSF in the contralateral hemisphere.

15. The system of claim 12, wherein the CT scans comprises a first CT scan and a second CT scan, the first CT scan being acquired at an earlier time than the second CT scan, and the instructions program the processor to:
extract an image of the patient's brain from the CT scans by extracting a first image of the patient's brain from the first CT scan and extracting a second image of the patient's brain from the second CT scan; and
identify CSF in the extracted image of the patient's brain in the CT scans by identifying a first volume of CSF from the first image of the patient's brain and a second volume of CSF from the second image of the patient's brain.

16. The system of claim 15, wherein the instructions program the processor to determine at least one CSF metric by determining a change in CSF (ΔCSF) in the patient's brain over time by subtracting the first volume of CSF from the second volume of CSF.

17. The system of claim 12, wherein the instructions program the processor to identify CSF in the extracted image of the patient's brain by processing the extracted image of the patient's brain with a deep learning algorithm stored in the memory and trained to perform CSF segmentation.

18. The system of claim 11, wherein the instructions program the processor to determine a likelihood of a future malignant edema occurring in the patient based at least in part on the at least one CSF metric by processing the at least one CSF metric with a prediction algorithm stored in the memory.

19. The system of claim 18, wherein the prediction algorithm comprises a recurrent neural network employing a long short-term memory (LSTM) architecture.

20. The system of claim 11, wherein the instructions program the processor to determine at least one CSF metric by determining a plurality of CSF metrics and to determine a likelihood of a future malignant edema occurring in the patient by determining a likelihood of a future malignant edema occurring in the patient based at least in part on the plurality of CSF metrics.
